(19) United States
(12) Patent Application Publication
(10) Pub. No.: US 2014/0234239 A1
(43) Pub. Date: Aug. 21, 2014

(54) SUSPENDING MEDIUM FOR GLITTER OR PIGMENTS IN URETHANE (METH)ACRYLATES

(71) Applicant: O P I Products, Inc., (US)

(72) Inventor: Sunil J. Sirdesai, Irvine, CA (US)

(73) Assignee: O P I Products, Inc., North Hollywood, CA (US)

(21) Appl. No.: 13/772,091

(22) Filed: Feb. 20, 2013

(51) Int. Cl. A61K 8/25; A61Q 3/02

(52) U.S. Cl. CPC A61K 8/25; A61Q 3/02
USPC 424/61

(57) ABSTRACT

A composition for forming a gel coating on a nail. The composition including a base component having an amount of a curable resin and a monomer sufficient to form a gel coating when applied to a nail. The composition further including at least one additive capable of enhancing an appearance of the base component and a suspending medium comprising fumed silica. The fumed silica is provided in an amount sufficient to suspend the additive in the base component.

### BACKGROUND

1. Field

An embodiment of the invention is directed to a medium for suspending particles in a polyurethane acrylate/methacrylate oligomer gel system, more specifically a fumed silica medium for suspending glitter in a urethane (meth)acrylate gel system. Other embodiments are also described and claimed.

2. Background

Urethane (meth)acrylates have been used in the nail-care industry as coatings over fingernails. In the nail-care industry parlance, these coatings are called gels. These coatings are temporary and are meant to be replaced every two weeks. Earlier versions of gels had to be removed mechanically using files or drills. However, the latest generation of gels can be soaked off the nails using low boiling ketones like acetone or methyl ethyl ketone and therefore cause minimal to no damage to the nail plate. The latest generation of gels are used for ‘gel-manicure’, a term coined by the nail-care industry where these gels, which are pigmented, are substituted for nail polish. These gel-manicures have become tremendously popular because they last (wear) at least four times longer than traditional nail polish. The downside is that they take about 15 minutes to soak off in acetone.

Suspending glitters or pigments in urethane (meth)acrylate oligomers has always presented a challenge with respect to gels. Such challenges, however, were easily overlooked in the case of earlier gel systems because the gel was sold in jars. Since the gel was in a jar, it could be stirred with a spatula before application in order to re-suspend the settled glitters or pigment within the gel. Since the gel could be stirred with a spatula, it was not critical that the gel medium be capable of suspending the glitter or pigments for an extended period of time. It is now preferred, however, to sell the gel systems in traditional nail lacquer bottles, which have narrower openings than the jars and therefore do not allow for the gel to be stirred with a spatula while within the bottle. Thus, the glitters and pigments settle to the bottom. This drawback has prevented the nail-care industry from realizing the full potential of gel systems because traditional nail lacquer bottles are unsuitable for packaging gels and therefore it is difficult to provide a full suite of colors and glitters necessary for painting nails or decorating nails (nail art) to the consumer.

### SUMMARY

An embodiment of the invention is directed to a suspending medium for urethane (meth)acrylate gel system compositions designed to provide nail coatings on the nail of a user. The suspending medium may be used to suspend an additive such as a pigment or glitter within the gel system composition. In one embodiment, the suspending medium is fumed silica. The fumed silica may form bonds with the additive, e.g. glitter, thereby forming particles having a relatively large surface area. The large surface area allows the particles to remain suspended within the gel system composition, and in turn, suspend the additive within the gel system. Since the additive is suspended in the gel system composition and does not require the use of a spatula for resuspension, the composition can be packaged within a traditional nail lacquer bottle.

The above summary does not include an exhaustive list of all aspects of the embodiments disclosed herein. It is contemplated that the embodiments may include all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### DETAILED DESCRIPTION

In this section we shall explain several preferred embodiments of the invention. Whenever the ingredients, ranges and other aspects of the formulations described in the embodiments are not clearly defined, the scope of the embodiments is not limited only to the formulations shown, which are meant merely for the purpose of illustration. Also, while numerous details are set forth, it is understood that some embodiments may be practiced without these details. In other instances, well-known structures and techniques have not been shown in detail so as not to obscure the understanding of this description.

The instant invention is directed to a gel system composition designed to provide overlays (e.g., a nail coating) on the nail of a user. The gel system composition may include an additive, such as a glitter or pigment, so as to provide an aesthetically appealing look to the user's nail. As previously discussed, prior art gel systems were lacking in that they could not sufficiently suspend glitters or pigments and therefore needed to be packaged in jars large enough to allow the user to stir the system with a spatula prior to application. The use of large jars, however, does not provide a suitable means for display and/or distribution of the gel system to the consumer. The gel system composition disclosed herein therefore solves this problem by providing a gel system in which additives such as a glitter or a pigment can be suspended and do not need to be mixed using a spatula. The gel system can therefore be packaged in a typical nail lacquer bottle (e.g., a 5 mL to 20 mL size bottle), which has a narrower opening than the previously discussed jars.

In one embodiment, the gel system is a composition suitable for packaging within a typical nail lacquer bottle and includes a base component and a suspending medium. The suspending medium can be incorporated into the base component to facilitate suspension of an additive therein. The base component may be made of any combination of ingredients suitable for forming a gel coating on a nail bed of a user. Representatively, in one embodiment, the base component may include a curable resin(s), a monomer(s) and a photoinitiator(s).

The curable resin may be, but is not limited to, polyurethane acrylate/methacrylate oligomers, for example, urethane (meth)acrylate. In one embodiment, the composition may include the curable resin in any amount sufficient to form a gel overlay on a user's nail. For example, in one embodiment, the composition may include the curable resin in an amount of from about 30 percent to about 65 percent, for example, from about 35 percent to about 62 percent, or from 45.5 percent to 54 percent by weight of the total composition.

The monomer may be any type of monomer suitable for use within a gel system. In some cases, the monomer may also serve as a crosslinker within the gel system. For example, in one embodiment, the monomer may be, but is not limited to, one or more of triethylene glycol dimethacrylate, hydroxypropyl methacrylate, isobornyl methacrylate, isobornyl acrylate, hydroxyethyl methacrylate (HEMA), hema maleate, PEG-4 dimethacrylate or ethyl methacrylate. The composition may include the monomer in any amount sufficient to facilitate formation of a gel overlay on a user's nail. For example, in one embodiment, the composition may include the monomer in an amount of from about 20 percent to about 60 percent, for example, from about 24 percent to about 57 percent, or from 35 percent to 44 percent by weight of the total composition.

The photoinitiator may be any type of photoinitiator suitable for use within a gel system. For example, in one embodiment, the photoinitiator may be, but is not limited to, one or more of benzoyl isopropanol, trimethylbenzoyl diphosphine oxide, trimethylbenzoyl diphenylphosphine oxide, hydroxycyclohexyl phenyl ketone, or ethyl trimethylbenzoylphenylphosphinate. The composition may include the photoinitiator in any amount sufficient to facilitate formation of a gel overlay on a user's nail. For example, in one embodiment, the composition may include the photoinitiator in an amount of from about 1 percent to about 10 percent, for example, from about 5 percent to about 6 percent, or about 5.5 percent by weight of the total composition.

In some embodiments, in addition to the curable resin, monomer and photoinitiator, the base component may include a thixotrope(s), a solvent(s), a plasticizer(s), a pigment(s) and/or a colorant(s). A suitable thixotrope may include, but is not limited to, polyquaternium 37. The thixotrope may be provided in any amount sufficient to facilitate formation of a coating on a user's nail, for example, from about 2 percent to about 10 percent, or from about 4 percent to 6 percent, or at least 5 percent by weight of the total composition.

A suitable solvent may include, but is not limited to, propylene glycol dicaprylate. The solvent may be provided in any amount sufficient to facilitate formation of a coating on a user's nail, for example, from about 0.5 percent to about 10 percent, or from about 1 percent to 5 percent, or at least 2 percent.

A suitable plasticizer may include, but is not limited to, PPG-1 trideceth 6 or dimethicone. The plasticizer may be provided in any amount sufficient to facilitate formation of a coating on a user's nail, for example, from about 0.5 percent to 10 percent, or from about 1 percent to 5 percent, or at least 2 percent by weight of the total composition.

Suitable pigments may include, but are not limited, titanium dioxide, iron oxide, red 21 or any other pigment desired and in any amount sufficient to enhance or modify an appearance of the base component. For example, the pigment may be provided in an amount of, for example, from about 0.5 percent to about 10 percent, or from about 1 percent to 5 percent, or from 2 percent to 3 percent by weight of the total composition.

Exemplary colorants may include, but are not limited to, violet 2, red 30 or any other colorant desired and in any amount sufficient to enhance or modify an appearance of the base component. For example, the colorant may be provided in an amount of, for example, from about 0 percent to about 5 percent, or from about 0.1 percent to 3 percent, or from 0.3 percent to 1 percent by weight of the total composition.

The suspending medium may be used to suspend an additive such as a glitter or a pigment within the base component so that the composition does not need to be vigorously stirred prior to application to the nail. In one embodiment, the suspending medium may be fumed silica. Fumed silica includes microscopic droplets of amorphous silica fused into branched, chainlike, three-dimensional secondary particles which then agglomerate into tertiary particles. The resulting powder has an extremely low bulk density and high surface area. It is believed that due to the extremely large surface area of the fumed silica particles, the hydroxyl groups on their sides form coordinate bonds or bonds via London forces with the cured resin (e.g., urethane (meth)acrylate) as well as with the additive (e.g., glitter or pigment). For example, glitter is a polyethylene terephthalate based compound which can form bonds with the hydroxyl groups of the fumed silica particles. The result is that the glitter is held in place within the base component by the fumed silica by creating a physical obstruction and chemically by forming bonds with functional groups. In particular, the large number of fumed silica particles with large surface areas prevents the glitter (or pigment) from settling within the base component. In addition, it was surprisingly found that even though the coordinate bond between glitter and fumed silica is somewhat weaker as compared to a pigment, the fumed silica was capable of suspending the glitter within the base component.

The fumed silica may be added in an amount sufficient to suspend the additive (e.g., glitter or pigment) within the base component. It is further noted that since the gel system composition is to be packaged within a traditional nail lacquer bottle (e.g., a 5 mL to 20 mL bottle), it should have a relatively low viscosity, as compared to gel systems packaged in large jars. In this aspect, the amount of fumed silica must be less than that which would increase a viscosity of the gel system composition beyond that suitable for use in a traditional nail lacquer bottle. For example, it has been found that the incorporation of 5 percent or more fumed silica within a composition results in a gel system having a viscosity or thickness too high for use in a traditional nail lacquer bottle. Thus, in one embodiment, the fumed silica is present in the composition in an amount less than 5 percent, for example, an amount of 2 percent or less, for example, about 1.5 percent or less, more specifically from about 1 percent to about 0.5 percent by weight of the total composition. It was unexpectedly found that as little as 0.5 percent fumed silica can sufficiently suspend the glitter within the composition through bonding between the glitter and fumed silica while still maintaining a relatively low viscosity sufficient to package the gel system in a traditional nail lacquer bottle.

The composition may include an additive such as glitter or pigment in any amount sufficient to enhance an appearance of the base component and/or provide the aesthetic characteristics desired. For example, in one embodiment, the composition may include glitter in an amount of from about 0.5 percent to about 5 percent, or from about 1 percent to about 4 percent, representatively, from 2.5 percent to 3.5 percent, for example, 3 percent by weight of the total composition.

In addition, it is further to be understood that the fumed silica is used in the gel system composition disclosed herein as a suspending medium. It is not added for the purpose of thickening the composition. Also, since the fumed silica is being used in the context of a gel system, suspending agents such as alumino silicates (clays) which are typically used in nail lacquer compositions to facilitate pigment suspension, are not used. In particular, in nail lacquer compositions fumed silica alone cannot suspend pigment or glitter particles. Rather, an inorganic agent called a ‘clay’, which is essentially an alumino silicate, must be added to the nail lacquer composition to help suspend any additives. In the gel system composition disclosed herein, fumed silica alone, in the absence of an additional suspending agent such as alumino silicates, is used to suspend the additive.

Exemplary gel system formulations are provided below.

### Example 1

| | | Weight |
|---|---|---|
| Chemical Name | Function | Percent(%) |
| Polyurethane Acrylate/Methacrylate | Curable Resin | 56 |
| Oligomers | | |
| Triethylene Glycol Dimethacrylate | Monomer/ | 24 |
| | Crossslinker | |
| Benzoyl Isopropanol | Photoinitiator | 5 |
| Trimethylbenzoyl Diphosphine Oxide | Photoinitiator | 1 |
| Polyquaternium 37 | Thixotrope | 5 |
| Propylene Glycol Dicaprylate | Solvent | 2 |
| PPG-1 Trideceth 6 | Plasticizer | 2 |
| Titanium Dioxide | Pigment | 1 |
| Fumed Silica | Suspending Aid | 1 |
| Glitter | Additive | 3 |

### Example 2

| | | Weight |
|---|---|---|
| Chemical Name | Function | Percent(%) |
| Polyurethane Acrylate/Methacrylate | Curable Resin | 49.5 |
| Oligomers | | |
| Hydroxypropyl Methacrylate | Monomer | 11 |
| Isobornyl Methacrylate | Monomer | 10 |
| HEMA | Monomer | 10 |
| Triethylene Glycol Dimethacrylate | Monomer/ | 8 |
| | Crosslinker | |
| Hydroxycyclohexyl Phenyl Ketone | Photoinitiator | 5 |
| Fumed Silica | Suspending Aid | 2 |
| Ethyl Trimethylbenzoylphenyl- | Photoinitiator | 1 |
| phosphinate | | |
| Glitter | Additive | 3.5 |

### Example 3

| | | Weight |
|---|---|---|
| Chemical Name | Function | Percent(%) |
| Polyurethane Acrylate/Methacrylate | Curable Resin | 62.5 |
| Oligomers | | |
| Triethyleneglycol Dimethacrylate | Monomer/ | 22 |
| | Crosslinker | |
| Fumed Silica | Suspending Aid | 0.5 |
| HEMA | Monomer | 7 |
| Hydroxycyclohexyl Phenyl Ketone | Photoinitiator | 3 |
| Benzoyl Isopropanol | Photoinitiator | 2.5 |
| Glitter | Additive | 2.5 |

### Example 4

| | | Weight |
|---|---|---|
| Chemical Name | Function | Percent(%) |
| Polyurethane Acrylate/Methacrylate | Curable Resin | 35 |
| Oligomers | | |
| Hydroxypropyl Methacrylate | Monomer | 29 |
| Isobornyl Methacrylate | Monomer | 23 |
| Fumed Silica | Suspending Aid | 1 |
| Hema Maleate | Monomer | 5 |
| Benzoyl Isopropanol | Photoinitiator | 5 |
| Ethyl Trimethylbenzoylphenyl- | Photoinitiator | 1 |
| phosphinate | | |
| Glitter | Additive | 1 |

### Example 5

| | | Weight |
|---|---|---|
| Chemical Name | Function | Percent(%) |
| Polyurethane Acrylate/Methacrylate | Curable Resin | 61.9 |
| Oligomers | | |
| PEG-4 Dimethacrylate | Monomer/ | 17 |
| | Crosslinker | |
| Ethyl Methacrylate | Monomer | 10 |
| Hydroxycyclohexyl Phenyl Ketone | Photoinitiator | 5 |
| Trimethylbenzoyl Diphenylphosphine | Photoinitiator | 1 |
| Oxide | | |
| Fumed Silica | Suspending Aid | 1 |
| Violet 2 | Colorant | 0.1 |
| Glitter | Additive | 4 |

### Example 6

| | | Weight |
|---|---|---|
| Chemical Name | Function | Percent(%) |
| Polyurethane Acrylate/Methacrylate | Curable Resin | 54.2 |
| Oligomers | | |
| Triethylene Glycol Dimethacrylate | Monomer/ | 30 |
| | Crosslinker | |
| Fumed Silica | Suspending Aid | 1 |
| HEMA | Monomer | 6 |
| Hydroxycyclohexyl Phenyl Ketone | Photoinitiator | 2.5 |
| Benzoyl Isopropanol | Photoinitiator | 2.5 |
| Ethyl Trimethylbenzoylphenyl- | Photoinitiator | 1 |
| phosphinate | | |
| Red 30 | Colorant | 0.3 |
| Glitter | Additive | 2.5 |

### Example 7

| | | Weight |
|---|---|---|
| Chemical Name | Function | Percent(%) |
| Polyurethane Acrylate/Methacrylate | Curable Resin | 45.5 |
| Oligomers | | |
| Triethylene Glycol Dimethacrylate | Monomer/ | 30 |
| | Crosslinker | |
| Isobornyl Acrylate | Monomer | 10 |
| HEMA | Monomer | 3 |
| Fumed Silica | Suspending Aid | 1.5 |
| Hydroxycyclohexyl Phenyl Ketone | Photoinitiator | 5 |
| Trimethylbenzoyl Diphenylphosphine | Photoinitiator | 1 |
| Oxide | | |
| Isobornyl Methacrylate | Monomer | 1 |
| Glitters | Additives | 3 |

### Example 8

| | | Weight |
|---|---|---|
| Chemical Name | Function | Percent(%) |
| Polyurethane Acrylate/Methacrylate | Curable Resin | 54 |
| Oligomers | | |
| HEMA | Monomer | 15 |
| Isobornyl Acrylate | Monomer | 15 |
| Hydroxycyclohexyl Phenyl Ketone | Photoinitiator | 2 |
| Benzoyl Isopropanol | Photoinitiator | 2 |
| Ethyl Trimethylbenzoylphenyl- | Photoinitiator | 1 |
| phosphinate | | |
| Dimethicone | Plasticizer | 1 |
| Isobornyl Methacrylate | Monomer | 4 |
| Glitters | Additives | 5 |
| Fumed Silica | Suspending Aid | 1 |

### Example 9

| | | Weight |
|---|---|---|
| Chemical Name | Function | Percent(%) |
| Polyurethane Acrylate/Methacrylate | Curable Resin | 52 |
| Oligomers | | |
| Triethylene Glycol Dimethacrylate | Monomer/ | 30 |
| | Crosslinker | |
| Fumed Silica | Suspending Aid | 1 |
| HEMA | Monomer | 5 |
| Benzoyl Isopropanol | Photoinitiator | 2 |
| Hydroxycyclohexyl Phenyl Ketone | Photoinitiator | 2 |
| Ethyl Trimethylbenzoylphenyl- | Photoinitiator | 1.5 |
| phosphinate | | |
| Titanium Dioxide | Pigment | 1 |
| Iron Oxide | Pigment | 1 |
| Red 21 | Pigment | 1 |
| Glitters | Additive | 3.5 |

### Example 10

| | | Weight |
|---|---|---|
| Chemical Name | Function | Percent(%) |
| Polyurethane Acrylate/Methacrylate | Curable Resin | 60 |
| Oligomers | | |
| Hydroxypropyl Methacrylate | Monomer | 16.5 |
| HEMA | Monomer | 15 |
| Hydroxycyclohexyl Phenyl Ketone | Photoinitiator | 4.5 |
| Trimethylbenzoyl Diphenylphosphine | Photinitiator | 1.5 |
| Oxide | | |
| Fumed Silica | Suspending Aid | 2 |
| Glitters | Additive | 0.5 |

A composition suitable for forming a gel overlay on a nail including any of the foregoing formulations may be formed according to any suitable technique. Representatively, in one embodiment, a curable resin(s), a monomer(s), a photoinitiator(s), a thixotrope(s), a solvent(s), a plasticizer(s) and/or a pigment(s) or colorant(s) may be mixed to form the base component. The suspending medium may be formed by mixing the suspending aid (i.e., fumed silica) with an additive (e.g., glitter). The suspending medium may then be added to the base component to form a gel system composition having the additive suspended therein. Alternatively, the composition may be formed by mixing any one or more of the ingredients of any of the above-referenced exemplary formulations together in any order and in any combination. The resulting composition may be divided into aliquots suitable for packaging within traditional nail lacquer bottles according to any suitable technique.

In the foregoing specification, the invention has been described with reference to specific embodiments thereof. It will, however, be evident that various modifications and changes can be made thereto without departing from the broader spirit and scope of the invention as set forth in the appended claims. For example, although the composition as described as being used to form a gel overlay on a nail, the composition may be any type of gel system in which suspension of an additive such as a glitter or pigment is desired. For example, the gel system may be used to form nail enhancements (e.g. artificial nails) on the nail. The specification is, accordingly, to be regarded in an illustrative rather than a restrictive sense.

1. A composition for forming a gel coating on a nail comprising:
a base component comprising an amount of a curable resin and a monomer sufficient to form a gel coating when applied to a nail;
at least one additive capable of enhancing an appearance of the base component, wherein the additive comprises glitter; and
a suspending medium comprising fumed silica, wherein the fumed silica is provided in an amount sufficient to suspend the additive in the base component.

2. The composition of claim 1 wherein the curable resin is urethane (meth)acrylate.

3. The composition of claim 1 wherein the additive is a first additive, the composition further comprising a second additive, wherein the second additive is a pigment.

4. The composition of claim 3 wherein the glitter and the curable resin are chemically bound to the fumed silica.

5. The composition of claim 1 wherein the fumed silica is provided in an amount of from 0.5 percent to 1.5 percent by weight of the total composition.

6. The composition of claim 2 wherein the amount of the urethane meth(acrylate) is from 30 percent to 65 percent by weight of the total composition.

7. The composition of claim 1 wherein the additive is suspended in the base component in the absence of an alumino silicate.

8. The composition of claim 1 wherein the gel coating forms a gel overlay which covers the entire nail.

9. The composition of claim 1 further comprising:
a photoinitiator in an amount of from 1 percent to 10 percent by weight of the total composition, and wherein the amount of the monomer is from 20 percent to 60 percent by weight of the total composition.

10. A composition for suspending a glitter or a pigment in a urethane (meth)acrylate gel comprising:
a suspending medium having an amount of fumed silica sufficient to suspend a glitter in a urethane (meth)acrylate gel composition.

11. The composition of claim 10 wherein the fumed silica is provided in an amount sufficient to suspend the glitter or the pigment without increasing a viscosity of the composition beyond a viscosity suitable for packaging of the composition in a nail lacquer bottle.

12. The composition of claim 10 wherein the suspending medium suspends the glitter in the absence of an alumino silicate component.

13. The composition of claim 10 wherein the urethane (meth)acrylate gel composition comprises urethane (meth)acrylate in an amount sufficient to form a gel overlay on a nail.

14. A composition for forming a gel coating on a nail comprising:
a base component comprising a curable resin in an amount of from 30 percent to 60 percent by weight of the total composition and a monomer in an amount of from 20 percent to 60 percent by weight of the total composition;
a suspending medium comprising fumed silica in an amount of from 0.5 percent to 1.5 percent by weight of the total composition; and
an additive in an amount of from 0.5 percent to 5 percent by weight of the total composition, and wherein the fumed silica suspends the additive in the base component.

15. The composition of claim 14 wherein the curable resin comprises polyurethane acrylate or methacrylate oligomers.

16. The composition of claim 14 wherein the additive comprises a glitter or a pigment.

17. The composition of claim 14 wherein the monomer is selected from the group consisting of triethylene glycol dimethacrylate, hydroxypropyl methacrylate, isobornyl methacrylate, isobornyl acrylate, hydroxyethyl methacrylate (HEMA), PEG-4 dimethacrylate, hema maleate and ethyl methacrylate.

18. The composition of claim 14 further comprising:
a photoinitiator selected from the group consisting of benzoyl isopropanol, trimethylbenzoyl diphosphine oxide, trimethylbenzoyl diphenylphosphine oxide, hydroxycyclohexyl phenyl ketone, ethyl trimethylbenzoylphenylphosphinate and benzoyl isopropanol.

19. The composition of claim 18 wherein the photoinitiator is in an amount of from 1 percent to 10 percent by weight of the total composition.
